(19) United States
(12) Patent Application Publication
(10) Pub. No.: US 2007/0135407 A1
(43) Pub. Date: Jun. 14, 2007

(54) Serm reduction of lipid profiles

(76) Inventor: Mitchell S. Steiner, Germantown, TN (US)

Correspondence Address: PEARL COHEN ZEDEK LATZER, LLP, 1500 BROADWAY 12TH FLOOR, NEW YORK, NY, 10036, US

(21) Appl. No.: 11/604,897

(22) Filed: Nov. 28, 2006

Related U.S. Application Data
(60) Provisional application No. 60/739,898, filed on Nov. 28, 2005.

(51) Int. Cl. A61K 31/55; A61K 31/445; A61K 31/40; A61K 31/138; A01N 31/14

(52) U.S. Cl. 514/212.01; 514/317; 514/408; 514/651; 514/720

(57) ABSTRACT

This invention is directed, inter alia to: 1)improving a lipid profile; 2) methods of reducing circulating lipid levels; 3) methods of increasing high density lipoprotein (HDL) levels; 4) methods of altering ratios of low density lipoprotein to high desnsity lipoprotein levels in a subject; 5) methods of treating atherosclerosis and its associated diseases; and 5) methods of treating ischemia in a male subject with prostate cancer having undergone androgen deprivation therapy; by administering to the subject a SERM compound of formula (I) and/or its N-oxide, ester, isomer, pharmaceutically acceptable salt, hydrate, or any combination thereof as described herein.

### CROSS REFERENCE TO RELATED APPLICATIONS

This Application claims the benefit of U.S. Provisional Application Ser. No. 60/739,898, filed Nov. 28, 2005, which is hereby incorporated by reference in its entirety.

### FIELD OF THE INVENTION

This invention is directed, inter alia to: 1) improving a lipid profile; 2) methods of reducing circulating lipid levels; 3) methods of increasing high density lipoprotein (HDL) levels; 4) methods of altering ratios of low density lipoprotein to high density lipoprotein levels in a subject; 5) methods of treating atherosclerosis and its associated diseases; and 5) methods of treating ischemia in a male subject with prostate cancer having undergone androgen deprivation therapy; by administering to the subject a SERM compound of formula (I-XI) and/or its N-oxide, ester, isomer, pharmaceutically acceptable salt, hydrate, or any combination thereof as described herein.

### BACKGROUND OF THE INVENTION

Cholesterol, triacylglycerol and other lipids are transported in body fluids by lipoproteins which may be classified according to their density, for example, the very low density lipoproteins (VLDL), intermediate density lipoproteins (IDL), low density lipoproteins (LDL) and high density lipoproteins (HDL).

It has been shown that high levels of LDL-Cholesterol in the blood correlate with atherosclerosis which is a progressive disease characterized in part by sedimentation of lipids in inner walls of arteries, particularly of coronary arteries. It has also been shown that a high blood level of LDL-Cholesterol correlates with coronary heart disease. Also, a negative correlation exists between blood levels of HDL cholesterol and coronary heart disease.

The level of total cholesterol in blood, which is the sum of HDL-Cholesterol, LDL-Cholesterol, VLDL-Cholesterol and chylomicron-Cholesterol, is not necessarily predictive of the risk of coronary heart disease and atherosclerosis.

The correlation between atherosclerosis and LDL cholesterol levels, however, is much higher than a similar correlation between atherosclerosis and total serum cholesterol levels.

Atherosclerosis is a disease characterized by the deposition of fatty substances, primarily cholesterol, and subsequent fibrosis in the arterial intima, resulting in plaque deposition on the inner surface of the arterial wall and degenerative changes within it. The ubiquitous arterial fatty plaque is the earliest lesion of atherosclerosis and is a grossly flat, lipid-rich atheroma consisting of macrophages and smooth muscle fibers. The fibrous plaque of the various forms of advanced atherosclerosis has increased intimal smooth muscle cells surrounded by a connective tissue matrix and variable amounts of intracellular and extracellular lipid.

Once the disease has progressed to the stage of significant persistent symptoms and compromised function, the next treatment step has conventionally been artery bypass grafting to repair or replace the damaged artery. While coronary artery bypass has become one of the more common major cardiovascular surgical procedures in the United States, surgery clearly is not the solution to the pathologic process because many patients are reluctant to accept its accompanying significant risk of morbidity and mortality. The autogenous veins or arteries used to bypass the disease-impaired arteries undergo atherosclerotic changes postoperatively generally at a faster rate than the original, affected arteries.

In addition, vascular blockage and cardiovascular disorders including myocardial infarction, coronary heart disease, hypertension and hypotension, cerebrovascular disorders including stroke, cerebral thrombosis and memory loss due to stroke;

peripheral vascular disease and intestinal infarction are caused by blockage of arteries and arterioles by atherosclerotic plaque. The production of atherosclerotic plaque formation is multi-factorial in its production. Hypercholesterolemia, especially elevated levels of low-density lipoprotein cholesterol (LDL) is an important risk factor for atherosclerosis, arteriosclerosis and associated diseases. Thus, and ideal treatment of high circulating lipid levels, and in particular, high ratios of LDL to HDL levels is currently lacking.

Prostate cancer is one of the most frequently occurring cancers among men in the United States, with hundreds of thousands of new cases diagnosed each year. Unfortunately, over sixty percent of newly diagnosed cases of prostate cancer are found to be pathologically advanced, with no cure and a dismal prognosis. One approach to this problem is to find prostate cancer earlier through screening programs and thereby reduce the number of advanced prostate cancer patients. Another strategy, however, is to develop drugs to prevent prostate cancer. One third of all men over 50 years of age have a latent form of prostate cancer that may be activated into the life-threatening clinical prostate cancer form. The frequency of latent prostatic tumors has been shown to increase substantially with each decade of life from the 50s (5.3-14%) to the 90s (40-80%). The number of people with latent prostate cancer is the same across all cultures, ethnic groups, and races, yet the frequency of clinically aggressive cancer is markedly different. This suggests that environmental factors may play a role in activating latent prostate cancer.

Toremifene is an example of a triphenylalkene compound described in U.S. Pat. Nos. 4,696,949 and 5,491,173 to Toivola et al., the disclosures of which are incorporated herein by reference. The parenteral and topical administration to mammalian subjects of formulations containing toremifene are described in U.S. Pat. No. 5,571,534 to Jalonen et al. and in U.S. Pat. No. 5,605,700 to DeGregorio et al., the disclosures of which are incorporated herein by reference.

Toremifene-containing formulations for reversing the multidrug resistance to cancer cells to a cytotoxic drug are described in U.S. Pat. No. 4,990,538 to Harris et al., the disclosure of which is incorporated herein by reference.

In addition to a need for an optimal treatment for prostate cancer, of the approaches to the treatment of prostate cancer, the most commonly utilized, androgen deprivation therapy, is fraught with significant side effects, including increase in total cholesterol, increase in tryglycerides, hot flashes, gynecomastia, osteoporosis, decreased lean muscle mass, depression and other mood changes, loss of libido, and erectile dysfunction [Stege R (2000), Prostate Suppl 10,38-42]. Consequently, complications of androgen blockade now contribute significantly to the morbidity, and in some cases the mortality, of men suffering from prostate cancer.

Given that more patients today are being treated by long-term androgen deprivation, high lipids levels in particular has become a clinically important side effect of men suffering from prostate cancer undergoing androgen deprivation. New innovative approaches are urgently needed at both the basic science and clinical levels to decrease the incidence of androgen-deprivation induced lipid profile changes or exacerbation in men suffering from prostate cancer.

### SUMMARY OF THE INVENTION

In one embodiment, this invention provides a method of reducing circulating lipid levels in a male subject with prostate cancer having undergone Androgen Deprivation Therapy (ADT), said method comprising administering to said subject a composition comprising a selective estrogen receptor modulator (SERM) compound or its pharmaceutically acceptable salt, hydrate, N-oxide, or any combination thereof.

In one embodiment this invention provides a method of reducing circulating lipid levels in a subject, wherein said method comprises administering to said subject a composition comprising a selective estrogen receptor modulator (SERM) compound of formula I or its pharmaceutically acceptable salt, hydrate, N-oxide, or any combination thereof, thereby reducing circulating lipid levels in a subject. 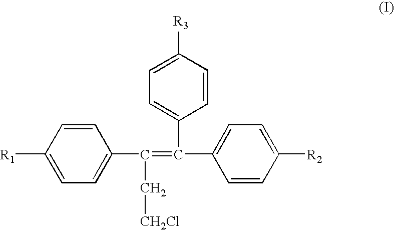
wherein R₁ and R₂, which can be the same or different, are H or OH, R₃ is OCH₂CH₂OH, OCH₂CH₂NR₄R₅, wherein R₄ and R₅, which can be the same or different, are H, an alkyl group of 1 to about 4 carbon atoms or forms together with the nitrogen a cyclic 5-8 membered ring; and their pharmaceutically acceptable carrier, diluents, salts, esters, or N-oxides, and mixtures thereof.

In one embodiment, this invention provides a method of treating atherosclerosis and its associated diseases including cardiovascular disorders, cerebrovascular disorders, peripheral vascular disorders, and intestinal vascular disorders in a subject comprising administering to said subject a composition comprising a selective estrogen receptor modulator (SERM) compound of formula I or its pharmaceutically acceptable salt, hydrate, N-oxide, or any combination thereof.

In one embodiment, this invention provides a method of treating ischemia in a tissue of a subject comprising administering to said subject a composition comprising a selective estrogen receptor modulator (SERM) compound or its pharmaceutically acceptable salt, hydrate, N-oxide, or any combination thereof. In some embodiments, the SERM compound is a compound of formula I, as herein described.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1 depicts circulating lipid levels of prostate cancer patients subjected to ADT, after treatment by toremifene.

### DETAILED DESCRIPTION OF THE PRESENT INVENTION

In some embodiments, this invention relates to methods of improving a lipid profile in a male subject. In one embodiment, the present invention provides 1) methods of reducing circulating lipid levels; 2) methods of increasing the high density lipoprotein (HDL) cholesterol levels; 3) methods of reducing the ratio of total circulating cholesterol to high density lipoprotein (HDL) levels; 4) methods of treating atherosclerosis and its associated diseases; 5) methods of treating ischemia 6) methods of improving lipid profiles in male subjects with prostate cancer having undergone ADT by administering to the subject a SERM compound of formula (I-XI) and/or its N-oxide, ester, isomer, pharmaceutically acceptable salt, hydrate, or any combination thereof as described herein.

In one embodiment, the phrase “Selective Estrogen Receptor Modulator” or “SERM” refers to a compound that affects estrogen receptor activity. In one embodiment, a SERM exhibits activity as an agonist, or, in another embodiment, as an antagonist, or in another embodiment, as a partial agonist, or in another embodiment, as a partial antagonist of the estrogen receptor. In one embodiment, the SERM exerts its effects on the estrogen receptor (e.g., ERα or ERβ) in a tissue-dependent manner.

In one embodiment, this invention provides for the use of a SERM as herein described to reduce circulating levels of low density lipoprotein (LDL) cholesterol. In one embodiment this invention provides for the use of a SERM as herein described to reduce circulating levels of triglycerides. In another embodiment, this invention provides for the use of a SERM as herein described to increase circulating levels of high density lipoprotein (HDL) cholesterol. In another embodiment, this invention provides for the use of a SERM as herein described to reduce the ratio of total circulating cholesterol to HDL levels.

In one embodiment, this invention provides a method of improving a lipid profile in a subject with prostate cancer having undergone or undergoing Androgen Deprivation Therapy (ADT), wherein said method comprises administering to said subject a composition comprising a selective estrogen receptor modulator (SERM) compound or its pharmaceutically acceptable salt, hydrate, N-oxide, or any combination thereof, thereby improving circulating lipid levels in a subject having prostate cancer and has undergone Androgen Deprivation Therapy (ADT).

In one embodiment, this invention provides a method of reducing circulating lipid levels in a subject, wherein said subject has prostate cancer and has undergone or is undergoing Androgen Deprivation Therapy (ADT), comprising administering to the subject a composition comprising a selective estrogen receptor modulator (SERM) compound or its pharmaceutically acceptable salt, hydrate, N-oxide, or any combination thereof.

In one embodiment, this invention provides a method of increasing circulating high density lipoprotein (HDL) cholesterol levels in a male subject with prostate cancer and having undergone or undergoing Androgen Deprivation Therapy (ADT), the method comprising administering to the subject a composition comprising a selective estrogen receptor modulator (SERM) compound or its pharmaceutically acceptable salt, hydrate, N-oxide, or any combination thereof.

In another embodiment, this invention provides a method of reducing the ratio of total circulating cholesterol to high density lipoprotein (HDL) levels in the subject, via administering a SERM to the subject.

In another embodiment, the SERM compound for use in the methods of this invention may be represented by the structure of formula I: 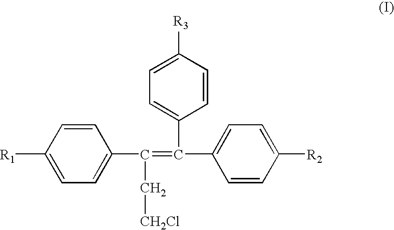
wherein R₁ and R₂, which can be the same or different, are H or OH, R₃ is OCH₂CH₂OH, OCH₂CH₂NR₄R₅, wherein R₄ and R₅, which can be the same or different, are H, an alkyl group of 1 to about 4 carbon atoms or forms together with the nitrogen a cyclic 5-8 membered ring; and their pharmaceutically acceptable carrier, diluents, salts, esters, or N-oxides, and mixtures thereof.

The term “alkyl” refers, in one embodiment, to a saturated aliphatic hydrocarbon, including straight-chain, branched-chain and cyclic alkyl groups. In one embodiment, the alkyl group has 1-12 carbons. In another embodiment, the alkyl group has 1-7 carbons. In another embodiment, the alkyl group has 1-6 carbons. In another embodiment, the alkyl group has 1-4 carbons. In another embodiment, the cyclic alkyl group has 3-8 carbons. In another embodiment, the cyclic alkyl group has 3-12 carbons. In another embodiment, the branched alkyl is an alkyl substituted by alkyl side chains of 1 to 5 carbons. In another embodiment, the branched alkyl is an alkyl substituted by haloalkyl side chains of 1 to 5 carbons. The alkyl group may be unsubstituted or substituted by a halogen, haloalkyl, hydroxyl, alkoxy carbonyl, amido, alkylamido, dialkylamido, cyano, CO2H, nitro, amino, alkylamino, dialkylamino, carboxyl, thio and/or thioalkyl.

In another embodiment, the SERM compound for use in the methods of this invention may comprise N,N-bis(4-hydroxyphenyl)-3,4-dimethylbenzamide; In another embodiment the SERM compound for use in the methods of this invention may comprise N,N-bis(4-hydroxyphenyl)-4-propylbenzamide; In another embodiment the SERM compound for use in the methods of this invention may comprise 3-fluoro-4-hydroxy-N-(4-hydroxyphenyl)-N-phenylbenzamide; In another embodiment the SERM compound for use in the methods of this invention may comprise N,N-bis(4-hydroxyphenyl)-4-pentylbenzamide. In another embodiment the SERM compound for use in the methods of this invention may comprise toremifene. In another embodiment the SERM compound for use in the methods of this invention may comprise ospemifene.

In another embodiment, the SERM compound for use in the methods of this invention may comprise any SERM known in the art, including SERMs described herein, or combinations thereof, for example, tamoxifen, ospemifene, 4-hydroxytamoxifen, idoxifene, 4-hydroxytoremifene ospemifene, droloxifene, raloxifene, arzoxifene, bazedoxifene, PPT (1,3,5-Tris(4-hydroxyphenyl)-4-propyl-1H-pyrazole), DPN [2,3-bis(4-hydroxyphenyl)propanenitrile], lasofoxifene, pipendoxifene, EM-800, EM-652, nafoxidine, zindoxifene, tesmilifene, miproxifene phosphate, RU58,688, EM 139, ICI 164,384, ICI 182,780, clomiphene, MER-25, diethylstibestrol, coumestrol, genistein, GW5638, LY353581, zuclomiphene, enclomiphene, delmadinone acetate, tibolone, DPPE, (N,N-diethyl-2-{4-(phenylmethyl)-phenoxy}ethanamine), TSE-424, WAY-070, WAY-292, WAY-818, prinaberel, ERB-041, WAY-397, WAY-244, ERB-196, WAY-169122, MF-101, ERb-002, ERB-037, ERB-017, BE-1060, BE-380, BE-381, WAY-358, [18F]FEDNP, LSN-500307, AA-102, CT-101, CT-102, VG-101, and the like.

In one embodiment, SERM compounds for use according to the methods of this invention comprise administration of the SERM and/or its analog, derivative, isomer, metabolite, pharmaceutically acceptable salt, hydrate, N-oxide, prodrug, ester, polymorph, impurity or crystal or combinations thereof. In another embodiment, methods of this invention comprise administration of a composition comprising a SERM compound, as described herein, or, in another embodiment, a combination of an analog, derivative, isomer, metabolite, pharmaceutically acceptable salt, pharmaceutical product, hydrate, N-oxide, prodrug, polymorph, ester, impurity or crystal of the SERM compounds of the present invention.

In one embodiment, the term “isomer” includes, but is not limited to, optical isomers and analogs, structural isomers and analogs, conformational isomers and analogs, and the like.

In one embodiment, the term “isomer” is meant to encompass optical isomers of the SERM compound. It will be appreciated by those skilled in the art that the SERMs of the present invention contain at least one chiral center. Accordingly, the SERMs used in the methods of the present invention may exist in, and be isolated in, optically-active or racemic forms. Some compounds may also exhibit polymorphism. It is to be understood that the present invention encompasses any racemic, optically-active, polymorphic, or stereroisomeric form, or mixtures thereof, and use of these for any application is to be considered within the scope of this invention.

In one embodiment, the SERMs for use according to the methods of this invention are the pure (R)-isomers. In another embodiment, the SERMs are the pure (S)-isomers. In another embodiment, the SERMs are a mixture of the (R) and the (S) isomers. In another embodiment, the SERMs are a racemic mixture comprising an equal amount of the (R) and the (S) isomers. It is well known in the art how to prepare optically-active forms (for example, by resolution of the racemic form by recrystallization techniques, by synthesis from optically-active starting materials, by chiral synthesis, or by chromatographic separation using a chiral stationary phase), and such methods are to be considered as part of this invention. In some embodiments, synthesis of such racemic forms may be accomplished by methods described and exemplified herein, or via appropriate modification thereof, as will be understood by one skilled in the art

In one embodiment, the SERMs are the pure (E)-isomers. In another embodiment, the SERMs are the pure (Z)-isomers. In another embodiment, the SERMs are a mixture of the (E) and the (Z) isomers.

The invention includes use of “pharmaceutically acceptable salts” of the SERMs as described herein, which may be produced, in one embodiment, using an amino-substituted SERM and organic and inorganic acids, for example, citric acid and hydrochloric acid. In one embodiment, the pharmaceutically acceptable salt of a SERM compound comprising a piperidine ring is an HCl salt. In another embodiment, the pharmaceutically acceptable salt of a SERM compound comprising a pyrrolidine ring is an HCl salt. In another embodiment, the pharmaceutically acceptable salt of a SERM compound comprising a morpholine ring is an HCl salt. In another embodiment, the pharmaceutically acceptable salt of a SERM compound comprising a piperazine ring is an HCl salt.

Pharmaceutically acceptable salts can be prepared from the phenolic compounds, in other embodiments, by treatment with inorganic bases, for example, sodium hydroxide. In another embodiment, esters of the phenolic compounds can be made with aliphatic and aromatic carboxylic acids, for example, acetic acid and benzoic acid esters.

This invention provides, in some embodiments, derivatives of the SERM compounds may be used in the methods of this invention. In one embodiment, the term “derivatives” refers to ether derivatives, acid derivatives, amide derivatives, ester derivatives or others, as known in the art. In another embodiment, this invention further includes hydrates of the SERM compounds. In one embodiment, the term “hydrate” refers to hemihydrate, monohydrate, dihydrate, trihydrate or others, as known in the art.

This invention provides, in other embodiments, use of metabolites of the SERM compounds. In one embodiment, the term “metabolite” refers to any substance produced from another substance by metabolism or a metabolic process.

This invention provides, in other embodiments, use of the pharmaceutical products of the SERM compounds. The term “pharmaceutical product” refers, in other embodiments, to a composition suitable for pharmaceutical use (pharmaceutical composition), for example, as described herein.

Compositions:

In some embodiments, this invention provides for the use of a composition comprising a SERM and may optionally include additional therapeutic agents. In some embodiments, such compositions may comprise compounds I, in any form or embodiment as described herein. In some embodiments, any of the compositions may consist of compounds I, in any form or embodiment as described herein. In some embodiments, of the compositions of this invention may consist essentially of compounds I, in any form or embodiment as described herein. In some embodiments, the term “comprise” refers to the inclusion of the indicated active agent, such as the compounds I, as well as inclusion of other active agents, and pharmaceutically acceptable carriers, excipients, emollients, stabilizers, etc., as are known in the pharmaceutical industry. In some embodiments, the term “consisting essentially of” refers to a composition, whose only active ingredient is the indicated active ingredient, however, other compounds may be included which are for stabilizing, preserving, etc. the formulation, but are not involved directly in the therapeutic effect of the indicated active ingredient. In some embodiments, the term “consisting essentially of” may refer to components which facilitate the release of the active ingredient. In some embodiments, the term “consisting” refers to a composition, which contains the active ingredient and a pharmaceutically acceptable carrier or excipient.

In another embodiment, the invention provides for the use of a pharmaceutical composition comprising a SERM, as herein described, or its prodrug, analog, isomer, metabolite, derivative, pharmaceutically acceptable salt, polymorph, crystal, impurity, N-oxide, ester, hydrate or any combination thereof and a suitable carrier or diluent.

In some embodiments, SERMs used according to the methods of this invention may be administered at various dosages to a subject, which in one embodiment, is a human subject. In one embodiment, the SERM is administered at a dosage of 0.1-200 mg per day. In one embodiment, the SERM is administered at a dose of 0.1-10 mg, or in another embodiment, 0.1-25 mg, or in another embodiment, 0.1- 50 mg, or in another embodiment, 0.3-15 mg, or in another embodiment, 0.3-30 mg, or in another embodiment, 0.5-25 mg, or in another embodiment, 0.5-50 mg, or in another embodiment, 0.75-15 mg, or in another embodiment, 0.75-60 mg, or in another embodiment, 1-5 mg, or in another embodiment, 1- 20 mg, or in another embodiment, 3-15 mg, or in another embodiment, 1-30 mg, or in another embodiment, 30-50 mg, or in another embodiment, 30-75 mg, or in another embodiment, 100-2000 mg, or in another embodiment, 80 mg, or in another embodiment, 30 mg. In some embodiments, the SERMs may be administered at different dosages, as a function of time, or disease/symptom/condition severity, or age, or other factors, as will be appreciated by one skilled in the art.

The SERMs of this invention may be administered at various dosages. In one embodiment, the SERM is administered at a dosage of 1 mg. In another embodiment the SERM is administered at a dosage of 5 mg, or in another embodiment, 3 mg, or in another embodiment 10 mg, or in another embodiment 15 mg, or in another embodiment 20 mg, or in another embodiment 25 mg, or in another embodiment 30 mg, or in another embodiment 35 mg, or in another embodiment 40 mg, or in another embodiment 45 mg, or in another embodiment 50 mg, or in another embodiment 55 mg, or in another embodiment 60 mg, or in another embodiment 65 mg, or in another embodiment 70 mg, or in another embodiment 75 mg, or in another embodiment 80 mg, or in another embodiment 85 mg, or in another embodiment 90 mg, or in another embodiment 95 mg or in another embodiment 100 mg.

In one embodiment, the methods of this invention make use of the SERM as described at a dose as described herein, given orally, once a day.

While the methods of this invention provide for the administration of SERM compounds as the sole active pharmaceutical agent, they can also be used in combination with one or more other compound as described herein, and/or in combination with other agents used in the treatment and/or prevention of the diseases, disorders and/or conditions, as described herein, as will be understood by one skilled in the art. In another embodiment, the SERM compounds can be administered sequentially with one or more such agents to provide sustained therapeutic and prophylactic effects. In another embodiment, the SERM compounds may be administered via different routes, at different times, or a combination thereof. It is to be understood that any means of administering combined therapies are to be considered as part of this invention, in an embodiment thereof.

Suitable agents for use according to the methods of this invention include, but are not limited to, other SERMs as well as traditional estrogen agonists and antagonists. Representative agents useful in combination with the compounds of the invention include, for example, tamoxifen, 4-hydroxytamoxifen, idoxifene, toremifene, 4-hydroxytoremifene ospemifene, droloxifene, raloxifene, arzoxifene, bazedoxifene, PPT (1,3,5-Tris(4-hydroxyphenyl)-4-propyl-1H-pyrazole), DPN [2,3-bis(4-hydroxyphenyl)propanenitrile], lasofoxifene, pipendoxifene, EM-800, EM-652, nafoxidine, zindoxifene, tesmilifene, miproxifene phosphate, RU58,688, EM 139, ICI 164,384, ICI 182,780, clomiphene, MER-25, diethylstibestrol, coumestrol, genistein, GW5638, LY353581, zuclomiphene, enclomiphene, delmadinone acetate, tibolone, DPPE, (N,N-diethyl-2-{4-(phenylmethyl)-phenoxy}ethanamine), TSE-424, WAY-070, WAY-292, WAY-818, prinaberel, ERB-041, WAY-397, WAY-244, ERB-196, WAY-169122, MF-101, ERb-002, ERB-037, ERB-017, BE-1060, BE-380, BE-381, WAY-358, [18F]FEDNP, LSN-500307, AA-102, CT-101, CT-102, VG-101, and the like.

In some embodiments, other agents that can be combined with one or more of the compounds of the invention include aromatase inhibitors such as, but not limited to, letrozole, anastrazole, atamestane, fadrozole, minamestane, exemestane, plomestane, liarozole, NKS-01, vorozole, YM-511, finrozole, 4-hydroxyandrostenedione, aminogluethimide, rogletimide.

In some embodiments, other agents useful for combination with the compounds of the invention include, but are not limited to antineoplastic agents, such as alkylating agents. In some embodiments, the SERMs of this invention may be administered in conjunction with antibiotics, hormonal antineoplastics and/or antimetabolites. Examples of useful alkylating agents include alkyl sulfonates such as busulfan, improsulfan and piposulfan; aziridines, such as a benzodizepa, carboquone, meturedepa and uredepa; ethylenimines and methylmelamines such as altretamine, triethylenemelamine, triethylenephosphoramide, triethylenethiophosphoramide and trimethylolmelamine; nitrogen mustards such as chlorambucil, chlomaphazine, cyclophosphamide, estramustine, iphosphamide, mechlorethamine, mechlorethamine oxide hydrochloride, melphalan, novembichine, phenesterine, prednimustine, trofosfamide, and uracil mustard; nitroso ureas, such as carmustine, chlorozotocin, fotemustine, lomustine, nimustine, ranimustine, dacarbazine, mannomustine, mitobronitol, mitolactol and pipobroman. More such agents will be known to those having skill in the medicinal chemistry and oncology arts.

In some embodiments, other agents suitable for combination with the compounds of the present invention include protein synthesis inhibitors such as abrin, aurintricarboxylic acid, chloramphenicol, colicin E3, cycloheximide, diphtheria toxin, edeine A, emetine, erythromycin, ethionine, fluoride, 5-fluorotryptophan, fusidic acid, guanylyl methylene diphosphonate and guanylyl imidodiphosphate, kanamycin, kasugamycin, kirromycin, and O-methyl threonine, modeccin, neomycin, norvaline, pactamycin, paromomycin, puromycin, ricin, α-sarcin, shiga toxin, showdomycin, sparsomycin, spectinomycin, streptomycin, tetracycline, thiostrepton and trimethoprim. Inhibitors of DNA synthesis, including alkylating agents such as dimethyl sulfate, mitomycin C, nitrogen and sulfur mustards, MNNG and NMS; intercalating agents such as acridine dyes, actinomycins, adriamycin, anthracenes, benzopyrene, ethidium bromide, propidium diiodide-intertwining, and agents such as distamycin and netropsin, can also be combined with compounds of the present invention in pharmaceutical compositions. DNA base analogs such as acyclovir, adenine, β-1-D-arabinoside, amethopterin, aminopterin, 2-aminopurine, aphidicolin, 8-azaguanine, azaserine, 6-azauracil, 2′-azido-2′-deoxynucliosides, 5-bromodeoxycytidine, cytosine, β-1-D-arabinoside, diazooxynorleucine, dideoxynucleosides, 5-fluorodeoxycytidine, 5-fluorodeoxyuridine, 5-fluorouracil, hydroxyurea and 6-mercaptopurine also can be used in combination therapies with the compounds of the invention. Topoisomerase inhibitors, such as coumermycin, nalidixic acid, novobiocin and oxolinic acid, inhibitors of cell division, including colcemide, colchicine, vinblastine and vincristine; and RNA synthesis inhibitors including actinomycin D, α-amanitine and other fungal amatoxins, cordycepin (3′-deoxyadenosine), dichlororibofuranosyl benzimidazole, rifampicine, streptovaricin and streptolydigin also can be combined with the compounds of the invention to provide pharmaceutical compositions. In some embodiments, immunostimulatory agents are co-administered with the SERMs of this invention.

In some embodiments, other agents suitable for combination with the compounds of the present invention include cardiac medications such as angiotensin converting enzyme (ACE) inhibitors such as enalapril, lisinopril, captopril, accupril, quinapril, benazopril and fosinopril. Antiarrhythmics such as amiodarone, sotalol, disopyramide, digoxin, procainamide and quinidine. Blood thinners such as aspirin (ASA), warfarin, clopidogrel and ticlopidine. Beta blockers such as atenolol, metoprolol, propranolol, sotalol, bisoprolol, carvedilol, timolol, nadolol, betaxolol pindolol and labetolol. Cholesterol lowering medications such as lovastatin, simvastatin, atorvostatin, pravastatin, niacin, cholestyramine and gemfibrozil. Nitrates such as isosorbide dinitrate and isosorbide mononitrate.

In one embodiment, other agents suitable for combination with the compounds of the present invention include hypertension medications such as angiotensin II receptor blockers such as losartan, valsartan, candesartan and irbesartan. Calcium channel blockers such as nifedipine, diltiazem, verapamil, amlodipine, felodipine, isradapine, nicardipene, nimodipine and Bepridil.

In one embodiment, other agents suitable for combination with the compounds of the present invention include stroke medications such as warfarin, clopidogrel, ticlopidine, dipyridamole, heparin and tissue plasminogen activator.

In addition, the compounds can be used, either singly or in combination as described above, in combination with other modalities for preventing or treating conditions, diseases or disorders, as described herein. In some embodiments, such other treatment modalities may include without limitation, surgery, radiation, hormone supplementation, diet regulation, wound debridement, etc., as will be appropriate for the condition being treated. These can be performed sequentially (e.g., treatment with a compound of the invention following surgery or radiation) or in combination (e.g., in addition to a diet regimen).

The additional active agents may generally be employed in therapeutic amounts as indicated in the PHYSICIANS' DESK REFERENCE (PDR) 53rd Edition (1999), which is incorporated herein by reference or such therapeutically useful amounts as would be known to one of ordinary skill in the art. The compounds of the invention and the other therapeutically active agents can be administered at the recommended maximum clinical dosage or at lower doses. Dosage levels of the active compounds in the compositions of the invention may be varied to obtain a desired therapeutic response depending on the route of administration, severity of the disease and the response of the patient. The combination can be administered as separate compositions or as a single dosage form containing both agents. When administered as a combination, the therapeutic agents can be formulated as separate compositions that are given at the same time or different times, or the therapeutic agents can be given as a single composition.

In one embodiment, this invention provides methods of treatment using a SERM or SERMs or composition/s of this invention. In one embodiment, the terms “treating” or “treatment”, includes preventative as well as disorder remitative treatment. The terms “reducing”, have their commonly understood meaning of lessening or decreasing, in another embodiment, or delaying, in another embodiment, or reducing, in another embodiment the incidence, severity or pathogenesis of a disease, disorder or condition. In embodiment, the term treatment refers to delayed progression of, prolonged remission of, reduced incidence of, or amelioration of symptoms associated with the disease, disorder or condition. In one embodiment, the terms “treating” “reducing”, refer to a reduction in morbidity, mortality, or a combination thereof, in association with the indicated disease, disorder or condition. In one embodiment, the methods of treatment of the invention reduce the severity of the disease, or in another embodiment, symptoms associated with the disease, or in another embodiment, reduces the number of biomarkers expressed during disease.

In one embodiment, the term “treating” and its included aspects, refers to the administration to a subject with the indicated disease, disorder or condition, or in some embodiments, to a subject predisposed to the indicated disease, disorder or condition. The term “predisposed to” is to be considered to refer to, inter alia, a genetic profile or familial relationship which is associated with a trend or statistical increase in incidence, severity, etc. of the indicated disease. In some embodiments, the term “predisposed to” is to be considered to refer to inter alia, a lifestyle which is associated with increased risk of the indicated disease. In some embodiments, the term “predisposed to” is to be considered to refer to inter alia, the presence of biomarkers which are associated with the indicated disease, for example, in cancer, the term “predisposed to” the cancer may comprise the presence of precancerous precursors for the indicated cancer.

The term “administering”, in another embodiment, refers to bringing a subject in contact with a compound of the present invention. Administration can be accomplished in vitro, i.e. in a test tube, or in vivo, i.e. in cells or tissues of living organisms, for example humans. In one embodiment, the present invention encompasses administering the compounds of the present invention to a subject.

Applications of the Compounds/Compositions:

In one embodiment, this invention provides methods of 1) improving the lipid profile of a subject; 2) reducing the circulating lipid levels in a subject; 3) increasing high density lipoprotein (HDL) cholesterol levels in a subject; 4) altering ratios of low density lipoprotein to high density lipoprotein levels in a subject; wherein said subject has prostate cancer and is undergoing or has undergone ADT, wherein said method comprises administering to said subject a composition comprising a SERM compound or its pharmaceutically acceptable salt, hydrate, N-oxide, or any combination thereof;. In another embodiment, the SERM, compound is of formula (I) or its prodrug, ester, analog, isomer, metabolite, derivative, pharmaceutically acceptable salt, pharmaceutical product, polymorph, crystal, impurity, N-oxide, hydrate or any combination thereof.

In another embodiment, the subject is undergoing or has undergone ADT. The terms “has undergone,” “undergoing,” and the like refer, in one embodiment, to subjects that have recently (within the last 6 months) or are currently receiving any treatment or therapy known in the art that reduces androgen levels in general or testosterone levels in particular. In another embodiment, the terms refer to a subject that received such a treatment or therapy more than 6 months previously. In one embodiment, the treatment or therapy is surgical. In another embodiment, the treatment or therapy is medical. In another embodiment, the treatment or therapy eliminates an androgen or a testosterone entirely, or below detectable levels. In another embodiment, the ADT is a side effect of a treatment or therapy not intended to reduce androgen or testosterone levels. Each of these possibilities represents a separate embodiment of the present invention.

In another embodiment, ADT is used for treating prostate cancer, for delaying the progression of prostate cancer, and for preventing and/or treating the recurrence of prostate cancer, which comprise administering LHRH analogs, reversible anti-androgens (such as bicalutamide or flutamide), anti-estrogens, anticancer drugs, 5-alpha reductase inhibitors, aromatase inhibitors, progestins, selective androgen receptor modulators (SARMS) or agents acting through other nuclear hormone receptors. In another embodiment, the ADT is administered monthly, or every 3, 4, 6 or 12 month. In another embodiment, the ADT is administered every two weeks in the first month, then every four weeks.

In some embodiments, this invention provides methods comprising administering a SERM compound to a subject that has prostate cancer and is undergoing or has undergone ADT. In one embodiment, the SERM compound of the present invention can be administered prior to the ADT. In another embodiment, the SERM compound of the present invention can be administered with the ADT. In another embodiment, the SERM compound of the present invention can be administered after the ADT.

In some embodiments, the methods of this invention comprise administering the SERM compound of this invention in combination with the ADT, prior to the ADT or after the ADT as a preventive for all diseases in this invention. In one embodiment the SERM is administered between 1-2 weeks before the ADT. In another embodiment the SERM is administered between 2-4 weeks prior to the ADT. In another embodiment the SERM is administered between 1-2 months before the ADT. In another embodiment the SERM is administered between 2-4 months before the ADT. In another embodiment the SERM is administered between 4-6 months before the ADT. In one embodiment the SERM is administered between 1-2 weeks after the ADT. In another embodiment the SERM is administered between 2-4 weeks after to the ADT. In another embodiment the SERM is administered between 1-2 months after the ADT. In another embodiment the SERM is administered between 2-4 months after the ADT. In another embodiment the SERM is administered between 4-6 months after the ADT.

In other embodiments, the present invention provides a method of treating any disease, disorder, or symptom associated with ADT. In other embodiments, the present invention provides a method of treating any disease, disorder, or symptom associated with androgen deprivation. In other embodiments, the present invention provides a method of treating any disease, disorder, or symptom associated with testosterone deprivation. Each disease, disorder, or symptom represents a separate embodiment of the present invention.

Cardiovascular cells, as well as reproductive tissues, bone, liver, and brain, express both of the known estrogen receptors, estrogen receptor-α (ER-α) and estrogen receptor-β (ER-β). These receptors are important targets for endogenous estrogen, estrogen replacement therapy (ERT), and pharmacological estrogen agonists. Estrogen-estrogen receptor complexes serve as transcription factors that promote gene expression with a wide range of vascular effects, including regulation of vasomotor tone and response to injury, which may be protective against development of atherosclerosis and ischemic diseases. Estrogen receptors in other tissues, such as the liver, may mediate both beneficial effects (e.g., changes in apoprotein gene expression that improve lipid profiles) and adverse effects (e.g., increases in gene expression of coagulation proteins and/or decreases in fibrinolytic proteins). Two general estrogen-mediated vascular effects are recognized. Rapid, transient vasodilation occurs within a few minutes after estrogen exposure, independently of changes in gene expression. Longer-term effects of estrogen on the vasculature, such as those related to limiting the development of atherosclerotic lesions or vascular injury, occur over hours to days after estrogen treatment and have as their hallmark alterations in vascular gene expression. Progesterone and other hormonal receptors are also expressed in the vasculature.

In one embodiment, this invention provides SERMs for improving the lipid profile and/or reducing the circulating lipid levels in a subject, wherein said subject has prostate cancer and has undergone ADT, or is undergoing ADT, and further suffers from one or more conditions selected from the group consisting of: atherosclerosis and its associated diseases, premature aging, Alzheimer's disease, stroke, toxic hepatitis, viral hepatitis, peripheral vascular insufficiency, renal disease, and hyperglycemia. In another embodiment the atherosclerosis and its associated diseases are selected from cardiovascular disorders, cerebrovascular disorders, peripheral vascular disorders, and intestinal vascular.

In some embodiments, lipid profiles are altered in a male subject having prostate cancer, as a result of ADT, producing a profile which is deleterious to the subject's well being or overall health. In some embodiments, the altered profile contributes, or is predictive, or is causative, in the development of a disease in the subject.

In one embodiment, the invention provides a method of treating, preventing, reducing the risk of mortality from cardiovascular and/or cerebrovascular disease in a subject, comprising administering a pharmaceutical composition comprising a SERM, compound of formula (I-XI) or its prodrug, ester, analog, isomer, metabolite, derivative, pharmaceutically acceptable salt, pharmaceutical product, polymorph, crystal, impurity, N-oxide, hydrate or any combination thereof. In another embodiment, the SERM compound is of formula XI.

In one embodiment, cardiovascular disease comprises, inter alia, atherosclerosis of the coronary arteries, angina pectoris, and myocardial infarction. In one embodiment, cerebrovascular disease comprises, inter alia, atherosclerosis of the intracranial or extracranial arteries, stroke, syncope, and transient ischemic attacks.

According to this aspect, and in one embodiment, the subject is a male subject with prostate cancer and is undergoing or has undergone ADT.

In another embodiment, this invention provides a method of 1) treating atherosclerosis; 2) treating cerebrovascular disorders; 3) treating peripheral vascular disorders; 4) treating intestinal vascular disorders comprising administering a pharmaceutical composition comprising a SERM, compound of formula (I) or its prodrug, ester, analog, isomer, metabolite, derivative, pharmaceutically acceptable salt, pharmaceutical product, polymorph, crystal, impurity, N-oxide, hydrate or any combination thereof. In another embodiment, the SERM compound is of formula I.

According to this aspect, and in one embodiment, the subject is a male subject with prostate cancer and is undergoing or has undergone ADT.

In one embodiment, the methods/compounds/compositions of this invention are useful in treating a subject having one or more risk factors for cardiovascular disease or cerebrovascular disease, such as hypercholesterolemia, hypertension, diabetes, cigarette smoking, familial or previous history of coronary artery disease, cerebrovascular disease, and cardiovascular disease. Hypercholesterolemia typically is defined as a serum total cholesterol concentration of greater than about 5.2 mmol/L (about 200 mg/dL). In some embodiments, risk factors may comprise hypertension (especially with systolic pressures greater than 160 mmHg), cardiac disease, transient ischemic attacks, diabetes mellitus, carotid bruits, and sickle cell disease. Obesity, a sedentary lifestyle, tobacco use, alcohol consumption, and elevated serum cholesterol and lipid levels may also be risk factors for cerebrovascular disease.

According to this aspect, and in one embodiment, the subject is a male subject with prostate cancer and is undergoing or has undergone ADT.

In one embodiment, the methods/compounds/compositions of this invention are useful in improving the lipid profile wherein the subject suffers from atherosclerosis. In another embodiment, the subject suffers from premature aging. In another embodiment, the subject suffers from a stroke. In another embodiment, the subject suffers from toxic hepatitis. In another embodiment, the subject suffers from viral hepatitis. In another embodiment, the subject suffers from viral hepatitis peripheral vascular insufficiency. In another embodiment, the subject suffers from renal disease. In another embodiment, the subject suffers from hyperglycemia.

According to this aspect, and in one embodiment, the subject is a male subject with prostate cancer and is undergoing or has undergone ADT.

In one embodiment this invention provides a method for a) treating, preventing, suppressing inhibiting atherosclerosis b) treating, preventing, suppressing inhibiting liver damage due to fat deposits comprising the step of administering to the subject a SERM/s as described herein and/or its analog, derivative, isomer, metabolite, pharmaceutically acceptable salt, pharmaceutical product, hydrate, N-oxide, ester, prodrug, polymorph, crystal, or any combination thereof, or a composition comprising the same, in an amount effective to treat, prevent or inhibit atherosclerosis and liver damage due to fat deposit.

According to this aspect, and in one embodiment, the subject is a male subject with prostate cancer and is undergoing or has undergone ADT.

In one embodiment, the SERM/s as described herein are useful in a) treating, preventing, suppressing, inhibiting, or reducing atherosclerosis.

In one embodiment atherosclerosis refers to a slow, complex disease that may begin with damage to the innermost layer of the artery. In another embodiment the causes of damage to the arterial wall may include a) elevated levels of cholesterol and in the blood; b) high blood pressure; c) tobacco smoke d) diabetes. In another embodiment, the condition is treatable in a smoker, despite the fact that tobacco smoke may greatly worsen atherosclerosis and speed its growth in the coronary arteries, the aorta and arteries in the legs. Similarly, in another embodiment, the methods of this invention may be useful in treating subjects with a family history of premature cardiovascular disease who have an increased risk of atherosclerosis.

In one embodiment, this invention provides method of treating, preventing, or ameliorating ischemia in a subject comprising administering to said subject a composition comprising a selective estrogen receptor modulator (SERM) compound or its pharmaceutically acceptable salt, hydrate, N-oxide, or any combination thereof. In one embodiment ischemia of any tissue may be thus treated in a subject comprising administering to said subject a composition comprising a selective estrogen receptor modulator (SERM) compound or its pharmaceutically acceptable salt, hydrate, N-oxide, or any combination thereof. In one embodiment, the tissue is a brain tissue. In another embodiment, the tissue is a gastrointestinal tissue. In another embodiment the tissue is a vascular tissue, or any combination thereof. According to this aspect, and in one embodiment, ischemia may be treated via the administration of the SERM compound of (I) as described herein and/or its analog, derivative, isomer, metabolite, pharmaceutically acceptable salt, pharmaceutical product, hydrate, N-oxide, ester, prodrug, polymorph, crystal, or any combination thereof.

In some embodiments, this invention provides for the use of a SERM as described herein, and/or compositions comprising the same, for treating any disease, disorder, or condition, in male subjects with prostate cancer, which arise as a result of ADT treatment in the subject, and are affected by the modulation of lipid profiles in the subject.

In some embodiments, such a disease, disorder, or condition may comprise stroke. In some embodiments, the term “stroke” refers, in other embodiments, to damage to nerve cells in the brain due to insufficient blood supply often caused by a bursting blood vessel or a blood clot. In some embodiments, such a disease, disorder, or condition may comprise heart disease. The term “heart disease”, in other embodiments, refers to a malfunction in the heart normal function and activity, including heart failure.

In another embodiment, the invention provides a method of treating or preventing peripheral vascular disease (PVD) in a male subject with prostate cancer, or having had prostate cancer, wherein the peripheral vascular disease arises as a result of ADT. According to this aspect and in one embodiment, the method comprises use of a SERM of this invention to regulate the Theological behaviour of blood thereby improving microcirculation. In another embodiment, the PVD may be caused by hypertension, as a consequence of lipid profiles promoting hypertension. In another embodiment, the PVD may be caused by a vaso-occlusive event, as a result of lipid profiles, which promote vaso-occlusion.

In one embodiment, the phrase “vaso-occlusive event” refers to an event that is characterized by or results in a decrease in the internal diameter of blood vessels either locally or systemically to an extent which impedes blood flow in a subject and/or is of a pathological nature. In one embodiment, a vaso-occlusive event encompasses pathological narrowing or occlusion of a stent, a vascular graft or a blood vessel. In one embodiment, the phrase, “pathological narrowing or occlusion” refers to narrowing or occlusion which is abnormal and/or disease-related. A vaso-occlusive event includes events which cause blood vessel narrowing or occlusion (such as thrombotic events, thromboembolic events and intimal hyperplasia) as well as conditions which result from such blood vessel narrowing (such as myocardial infarction and ischemic stroke).

Thrombotic events including thromboembolic events can be serious medical conditions particularly since they can cause a reduction in blood flow to critical organs including the brain and myocardium. Examples of thrombotic events include but are not limited to arterial thrombosis, including stent and graft thrombosis, cardiac thrombosis, coronary thrombosis, heart valve thrombosis and venous thrombosis. Cardiac thrombosis is thrombosis in the heart. Arterial thrombosis is thrombosis in an artery. Coronary thrombosis is the development of an obstructive thrombus in a coronary artery, often causing sudden death or a myocardial infarction. Venous thrombosis is thrombosis in a vein. Heart valve thrombosis is a thrombosis on a heart valve. Stent thrombosis is thrombosis resulting from and/or located in the vicinity of a vascular stent. Graft thrombosis is thrombosis resulting from and/or located in the vicinity of an implanted graft, particularly a vascular graft. It is to be understood that any of these conditions may arise as a result of inappropriate lipid profiles in the subject, as a result of ADT treatment.

Examples of conditions or disorders that result from thrombotic events include but are not limited to myocardial infarction, stroke, transient ischemic attacks, amaurosis fugax, aortic stenosis, cardiac stenosis, coronary stenosis and pulmonary stenosis. Stenosis is the narrowing or stricture of a duct or canal. Coronary stenosis is the narrowing or stricture of a coronary artery. Cardiac stenosis is a narrowing or diminution of any heart passage or cavity. Pulmonary stenosis is the narrowing of the opening between the pulmonary artery and the right ventricle. Aortic stenosis is narrowing of the aortic orifice of the heart or of the aorta itself.

Vaso-occlusive events also include disorders in which the blood vessel narrowing results not necessarily from a thrombus but rather a thickening of the vessel wall such as with intimal hyperplasia. Intimal hyperplasia refers to a condition characterized by abnormal proliferation of the cells of the intimal layer of the blood vessel wall.

Thus, one aspect of the invention relates to methods/compounds/compositions for reducing the risk of a thrombotic event. In one embodiment, the method reduces the risk of stroke. Stroke is a condition resulting from the lack of oxygen to the brain, resulting from one or more occlusive thrombi. Depending on the area of the brain affected, stroke can result in a wide range of symptoms from transient ischemic attacks to death (e.g., coma, reversible or irreversible paralysis, speech problems or dementia). In preferred embodiments, the stroke is non-hemorrhagic in nature.

The methods/compounds/compositions of the invention in other embodiments relates to reducing the risk of myocardial infarction. Myocardial infarction refers to an irreversible injury to the heart muscle. Myocardial infarction generally results from an abrupt decrease in coronary blood flow following a thrombotic occlusion (e.g., a thromboembolism) of a coronary artery. The thrombus, in many instances, forms after the rupture of atherosclerotic plaques in diseased coronary arteries. Such injury is highly correlated with factors such as cigarette smoking, hypertension and lipid accumulation.

Transient ischemic attack is a transient acute neurological dysfunction resulting from a thromboembolism in the cerebral circulation. Amaurosis fugax is the temporary monocular blindness resulting from a thromboembolism in the retinal vasculature.

The methods/compounds/compositions of the invention can be used to reduce the risk of a primary or a secondary vaso-occlusive event such as a thrombotic event or to inhibit the progression of such an event. A primary vaso-occlusive event refers to the first known vaso-occlusive event experienced by the subject. A secondary vaso-occlusive event refers to a vaso-occlusive event which occurs in a subject known or diagnosed as having previously experienced a vaso-occlusive event (i.e., a primary vaso-occlusive event).

It is to be understood that any of these conditions, predispositions, symptoms, byproducts, etc. of the same, may be positively affected by the administration of a compound/composition of this invention, and such use is to be considered an embodiment thereof. Further, in some embodiments of this invention, such conditions, predispositions, symptoms, byproducts, etc. of the same, are a result of the subject having undergone or undergoing ADT, and the conditions, predispositions, symptoms, byproducts, etc. of the same are positively affected by changes in circulating, or tissue lipid distribution, or ratios of lipids thereof.

### EXAMPLES

### Example 1

### Toremifene Lowers Total LDL Cholesterol and Triglycerides and Raises HDL on Prostate Cancer Patients on Androgen Deprivation Therapy (ADT)

Methods:

1392 men 50 years old or more, with histologically documented prostate cancer and receiving ADT were randomized to toremifene (80 mg/day) or placebo treated groups in a human clinical trial. An interim analysis evaluated changes in circulating lipid levels from baseline to month 12 in the first 197 subjects to complete their first year to determine changes in total cholesterol, low density lipoprotein (LDL) cholesterol, high density lipoprotein (HDL) cholesterol, triglycerides and the ratio of total circulating cholesterol to HDL levels in the respective subjects.

Results:

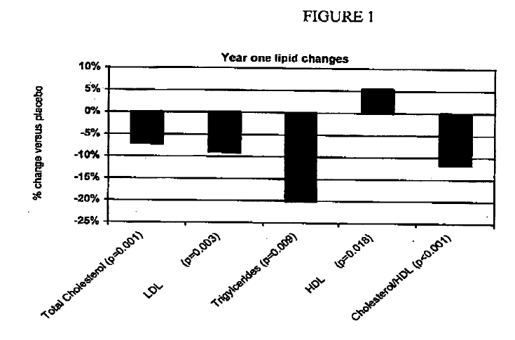

Prostate cancer patients having undergone Androgen Deprivation Therapy (ADT) who received toremifene were compared to placebo groups. Toremifene treatment resulted in lower total circulating cholesterol (−7.1%; p=0.001), LDL (−9.0%; p=0.003), and triglyceride (−20.1%; p=0.009) levels, a reduction in the total cholesterol/HDL ratio (−11.7%; p<0.001), and higher HDL levels (+5.4%; p=0.018) (FIG. 1).

Subjects concurrently administered Statins demonstrated further reduction of total cholesterol, yet the magnitude of lipid changes elicited by toremifene treatment was greater in patients who were not concomitantly taking statins. Accordingly, patients treated with toremifene had a statistically significant improvement in all serum lipid parameters measured.

### Example 2

### Exemplified SERM Compounds Lowering LDL Cholesterol Levels

Methods:

In addition to Toremifene, other SERM compounds may be similarly evaluated in clinical trial settings. The following compounds may be similarly administered as described in Example 1, and their effect in altering lipid profiles in subjects with prostate cancer, undergoing ADT may be similarly evaluated. Some of the compounds thus evaluated may comprise:
Compound (1): N,N-bis(4-hydroxyphenyl)-3,4-dimethylbenzamide;
Compound (2): N,N-bis(4-hydroxyphenyl)-4-propylbenzamide;
Compound (3): 3-fluoro-4-hydroxy-N-(4-hydroxyphenyl)-N-phenylbenzamide;
Compound (4): N,N-bis(4-hydroxyphenyl)-4-pentylbenzamide; and/or Ospemifene.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

What is claimed is:

1. A method of reducing circulating lipid levels in a male subject with prostate cancer having undergone Androgen Deprivation Therapy (ADT), said method comprising administering to said subject a composition comprising a selective estrogen receptor modulator (SERM) compound or its pharmaceutically acceptable salt, hydrate, N-oxide, or any combination thereof.

2. The method of claim 1, wherein said lipid levels, which are reduced comprise a triglyceride, low density lipoprotein (LDL) cholesterol, or a combination thereof.

3. The method of claim 1, wherein said method comprises increasing circulating levels of high density lipoprotein (HDL) cholesterol in said subject.

4. The method of claim 1, wherein said method further comprises reducing the ratio of total circulating cholesterol levels to high density lipoprotein (HDL) levels in said subject.

5. The method of claim 1, wherein said subject further suffers from atherosclerosis and its associated diseases, premature aging, Alzheimer's disease, stroke, toxic hepatitis, viral hepatitis, peripheral vascular insufficiency, renal disease, hyperglycemia, or any combination thereof.

6. The method of claim 1, wherein said selective estrogen receptor modulator compound is represented by the structure of formula I:

7. The method of claim 1, wherein said selective estrogen receptor modulator compound is represented by the structure of formula I: 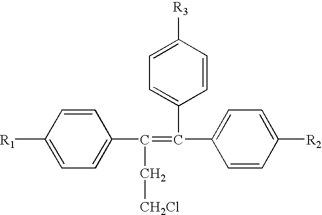
wherein R₁ and R₂, which can be the same or different, are H or OH, R₃ is OCH₂CH₂0H or OCH₂CH₂NR₄R₅, wherein R₄ and R₅, which can be the same or different, are H, an alkyl group of 1 to about 4 carbon atoms or forms together with the nitrogen a cyclic 5-8 membered ring; and their pharmaceutically acceptable carrier, diluents, salts, esters, or N-oxides, and mixtures thereof.

8. A method of reducing circulating lipid levels in a subject, said method comprising administering a composition comprising a selective estrogen receptor modulator (SERM) compound of formula XI or its pharmaceutically acceptable salt, hydrate, N-oxide, or any combination thereof; 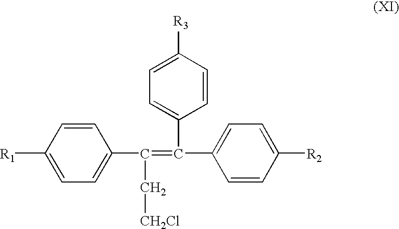
wherein R₁ and R₂, which can be the same or different, are H or OH, R₃ is OCH₂CH₂OH, OCH₂CH₂NR₄R₅, wherein R₄ and R₅, which can be the same or different, are H, an alkyl group of 1 to about 4 carbon atoms or forms together with the nitrogen a cyclic 5-8 membered ring; and their pharmaceutically acceptable carrier, diluents, salts, esters, or N-oxides, and mixtures thereof.

9. The method of claim 8, wherein said lipid is selected from triglycerides, low density lipoprotein (LDL) cholesterol, or combination thereof.

10. The method of claim 8, wherein circulating high density lipoprotein (HDL) cholesterol levels are increased in said subject.

11. The method of claim 8, wherein said method further reduces the ratio of total circulating cholesterol to high density lipoprotein (HDL) levels in said subject.

12. The method of claim 8, wherein said compound of formula XI is toremifene, its pharmaceutically acceptable carrier, diluents, salts, esters, or N-oxides, and mixtures thereof.

13. The method of claim 8, wherein said subject suffers from one or more conditions selected from the group consisting of: atherosclerosis, premature aging, Alzheimer's disease, stroke, toxic hepatitis, viral hepatitis, peripheral vascular insufficiency, renal disease, and hyperglycemia.

14. The method of claim 8, wherein said subject is healthy and seeks to optimize his or her health.

15. The method of claim 8, wherein said composition is a parenteral formulation comprises a liposome and a complex of said SERM compound and a cyclodextrin compound.

16. A method of treating atherosclerosis and its associated diseases including cardiovascular disorders, cerebrovascular disorders, peripheral vascular disorders, and intestinal vascular disorders in a subject comprising administering to said subject a composition comprising a selective estrogen receptor modulator (SERM) compound of formula I or its pharmaceutically acceptable salt, hydrate, N-oxide, or any combination thereof, 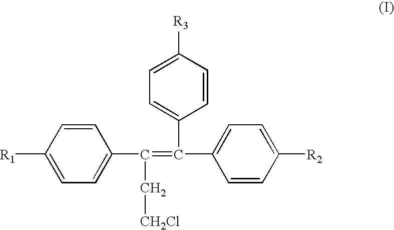
wherein
R₁ and R₂, which can be the same or different, are H or OH, R₃ is OCH₂CH₂OH, or OCH₂CH₂NR₄R₅, wherein R₄ and R₅, which can be the same or different, are H, an alkyl group of 1 to about 4 carbon atoms or forms together with the nitrogen a cyclic 5-8 membered ring; and their pharmaceutically acceptable carrier, diluents, salts, esters, or N-oxides, and mixtures thereof; thereby treating a subject having atherosclerosis and its associated diseases

17. The method of claim 16, wherein said compound of formula I is toremifene, its pharmaceutically acceptable carrier, diluents, salts, esters, or N-oxides, and mixtures thereof.

18. The method of claim 16, wherein said composition is a parenteral formulation comprises a liposome and a complex of said SERM compound and a cyclodextrin compound.

19. A method of treating ischemia in a tissue of a subject comprising administering to said subject a composition comprising a selective estrogen receptor modulator (SERM) compound or its pharmaceutically acceptable salt, hydrate, N-oxide, or any combination thereof.

20. The method of claim 19, wherein said tissue is a brain tissue, gastrointestinal tissue, vascular tissue, or any combination thereof.

21. The method of claim 19, wherein said selective estrogen receptor modulator compound is represented by the structure of formula I: 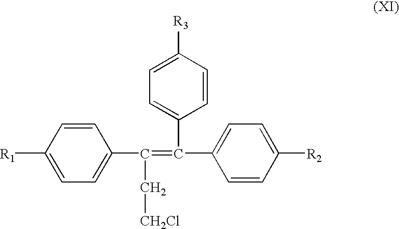
wherein R₁ and R₂, which can be the same or different, are H or OH, R₃ is OCH₂CH₂OH, OCH₂CH₂NR₄R₅, wherein R₄ and R₅, which can be the same or different, are H, an alkyl group of 1 to about 4 carbon atoms or forms together with the nitrogen a cyclic 5-8 membered ring; and their pharmaceutically acceptable carrier, diluents, salts, esters, or N-oxides, and mixtures thereof.
